(19) United States
(12) Patent Application Publication
(10) Pub. No.: US 2011/0236483 A1
(43) Pub. Date: Sep. 29, 2011

(54) RNA VECTOR THERAPY

(76) Inventors: LANE BERNARD SCHEIBER, Annandale, VA (US); Lane Bernard Scheiber, II, Grosse Ile, MI (US)

(21) Appl. No.: 12/748,433

(22) Filed: Mar. 28, 2010

(51) Int. Cl. A61K 9/14; A61K 31/713; C07K 14/00; C07K 14/435; C07H 21/02; A61P 43/00

(52) U.S. Cl. 424/484; 514/44 R; 530/350; 530/395; 536/23.1

(57) ABSTRACT

The innovative treatment strategy described here utilizes configurable microscopic medical payload delivery devices to act as a transport vector to deliver a wide variety of cellular ribonucleic acid molecules to specific types of cells in the body. Utilizing probes on the exterior of the transport devices, the transport devices locate a specific type of cell in the body. Once a specific target cell type has been encountered, the configurable microscopic medical payload delivery devices insert their payload of cellular ribonucleic acid molecules into the target cells. By delivering cellular ribonucleic acid molecules into specific cells a wide variety of protein deficiencies are correctable, gene expression is capable of being modulated, and telomere synthesis is enhanced.

### CROSS-REFERENCE TO RELATED APPLICATIONS: None.

### STATEMENT REGARDING SPONSORED RESEARCH OR DEVELOPMENT: None.

### REFERENCE TO SEQUENCE LISTING, A TABLE, OR COMPUTER LISTING COMPACT DISC APPENDIX: Not applicable.

©2010 Lane B. Scheiber and Lane B. Scheiber II. A portion of the disclosure of this patent document contains material which is subject to copyright protection. The copyright owners have no objection to the facsimile reproduction by anyone of the patent document or the patent disclosure, as it appears in the Patent and Trademark Office patent file or records, but otherwise reserves all copyright rights whatsoever.

### BACKGROUND OF THE INVENTION

1. Field of the Invention

This invention relates to any medical device intended to treat medical conditions utilizing a configurable microscopic medical payload delivery device to insert cellular ribonucleic acid molecules into one or more specific type of cells in the body to improve cell function.

2. Description of Background Art

Cellular ribonucleic acid (RNA) molecules are divided into two major functional categories which are ‘protein coding RNAs’ and ‘non-coding RNAs’. Protein coding RNAs, usually referred to as messenger RNAs, undergo the process of translation in the cytoplasm of the cell and produce proteins by ribosomes decoding the genetic information carried in the coding region of the messenger RNA. Non-coding RNAs perform a wide variety of tasks both in the nucleus and in the cytoplasm of a cell.

Present medical research is attempting to utilize viruses to deliver genetic information into cells. Research in the field of gene therapy has involved certain naturally occurring viruses. Some of the common viral vectors that have been investigated include: Adeno-associated virus, Adenovirus, Alphavirus, Epstein-Barr virus, Gammaretrovirus, Herpes simplex virus, Letivirus, Poliovirus, Rhabdovirus, Vaccinia virus. Naturally occurring virus vectors are limited to the naturally occurring external probes that are affixed to the outer wall of the virus. The external probes fixed to the outside wall of a virus virion dictate which type of cell the virus can engage and infect. Therefore, as an example, the function of the adenovirus, a respiratory virus, is strictly limited to engaging and infecting specific lung cells. Used as a medical treatment device, the adenovirus can only deliver gene therapy to specific lung cells, which severely limits this vector's usefulness as a deliver device. The therapeutic function of all naturally occurring viral vectors is limited to delivering a DNA or RNA payload to the cell type the viral vector naturally targets as its host cell.

Naturally occurring viruses also have the disadvantage of being susceptible to detection and elimination by a body's immune system. Viruses have been infecting humans for hundreds of thousands of years. A human's immune system is very efficient at detecting the presence of most naturally occurring viruses when such a virus is inside the body. The human immune system is quite capable of generating a vigorous response to most intruding viruses, attacking and neutralizing virus virions whenever a virus virion physically exists are outside the exterior wall of the virus's host cell. If gene therapy in its current state were to become a clinical therapeutic tool, the naturally occurring viruses selected for gene therapy research will have limited effectiveness due the fact that once the viral vector is introduced into the body, the body's the immune system will quickly engage and eliminate the viral vectors, possibly before the vector is able to deliver its payload to its host cell or target cell.

Cichutek, K., 2001 (U.S. Pat. No. 6,323,031 B1) teaches preparation and use of novel lentiviral SiVagm-derived vectors for gene transfer into selected cell types, specifically into proliferatively active and resting human cells.

Cichutek teaches that it is indeed plausible to re-configure an existing virus and use it as a transport vehicle, though Cichutek's specification and claims are too limited to describe a method that will work for all cell types, if indeed if it will work for any cell type.

Cichutek describes vectors for ‘gene transfer’; in the claims the language that is used is ‘genetic information’. Cichutek's claim 1 of the cited patent states ‘A propagation-incompetent SIVagm vector comprising a viral core and a viral envelope, wherein the viral core comprises a simian immunodeficiency virus (SIVagm) viral core of the African vervet monkey Chlorocebus.’ Cichutek's does not describe in his claims any further details of the intended payload other than the stating ‘SIVagm viral core’ in claim 1; in claims 5 & 6 Cichutek describes only ‘genetic information’. Transfer of ‘genetic information’ dramatically limits the useful application of Cichutek's patent in the treatment of medical diseases.

Cichutek does not claim the use of specific glycogen probes to target specific types of cells. Cichutek's approach is dependent upon the probes naturally present on the viral vectors reported in the patent, which will direct the viral vectors to only those cells the viruses naturally use as their host cell. Cichutek's approach is very restrictive, limited to gene transfer to only cells the viruses use as their natural host cell.

It is questionable that Cichutek's approach as described in the specification and claims is feasible. Cichutek's claim 4, states ‘The SIVagm vector of claim 1, wherein the viral envelope further comprises a single chain antibody (scFv) or a ligand of a cell surface molecule.’ By use of the words ‘a’ and ‘or’ in the claim, the claim is limited in the singular, meaning Cichutek claims a single chain antibody or a singular ligand. Singular type antibodies or ligands can be used for cell-to-cell communication, but to open an access portal into a cell and insert a payload into the cell requires two different types of antibodies or ligands. As an example human immunodeficiency virus requires the use of both the gp120 and gp41 probes to open a portal into a T-Helper cell and insert its viral genome into the T-Helper cell. The gp120 probe engages the CD4+ cell-surface receptor on the T-cell. Once the gp120 probe has successfully engaged a CD4+ cell-surface receptor on the target T-Helper cell, then the HIV virion's gp41 probe can engage either a CXCR4 or a CCR5 cell-surface receptor on the T-Helper cell in order to open up an access portal for HIV to insert its viral genome into a T-Helper cell. It is well documented in the medical literature that a genetic defect leading to an abnormality in the CXCR4 cell-surface receptor prevents HIV virions from opening an access portal and inserting its genetic payload into such T-Helper cells. This genetic defect in the CXCR4 cell-surface receptors offers the subset of people carrying the genetic defect resistance to HIV infection. This example demonstrates the need for at least two types of glycoprotein probes to be present on the surface of a viral vector in order for a viral vector to be capable of opening an access portal and delivering the payload the vector carries into its host cell or target cell.

A delivery system that offered a defined means of targeting specific types of cells would invoke minimal or no response by the innate immune system and the adaptable immune system when present in the body, and a delivery system that would be capable of inserting into cells a wide variety of ribonucleic acid molecules would significantly improve the current medical treatment options available to clinicians treating patients.

The solution to arriving at a versatile, workable delivery system that will meet the needs of a number of medical treatments involves three important elements. These elements include:

(1) configurable external probes whereby more than one type of protein structure probe or more than one type of glycoprotein probe is to be used to engage and access specific target cell types in order to successfully deliver a payload into a specific cell type,

(2) an exterior envelope comprised of a protein shell or lipid layer expressing the least number of cell-surface markers, such as the use of a stem cell to act as the host cell to manufacture the delivery devices,

(3) configuring the core of the vector to enable it to carry and deliver a wide variety of cellular ribonucleic acid molecules.

For purposes of this text, the use of the terms ‘specific target cell type’, ‘target cell’, ‘specific cell type’, ‘specific cell’, ‘specific type of cell’ are equivalent and interchangeable; the configuration of cell-surface receptors that a specific cell type has located on and protruding from its outer cell membrane determines the cell type.

Viruses are obligate parasites. Viruses simply represent a carrier of genetic material and by themselves viruses are unable to replicate or carry out any form of biologic function outside their host cell. A ‘virion’ refers to the physical structure of a single complete virus as it exists outside of the host cell; a more archaic term for ‘viral virion’ was ‘virus particle’. Viruses are generally comprised of one or more nested shells constructed of one or more layers of protein, some with a lipid outer envelope, a genetic payload that represents the instruction code necessary to replicate the virus, and protein enzymes to help facilitate the genetic payload in the function of replicating copies of the virus once the genetic payload has been delivered to a host cell. Located on the outer shell or envelope of a virus are probes. The function of a virus's external probes is to locate and engage a host cell's receptors. The virus's surface probes are designed to detect, make contact with and functionally engage one or more receptors located on the exterior of the type of cell that will offer the virus the proper environment in which to construct copies of itself. A host cell provides the virus the proper biologic machinery for the virus to successfully replicate itself. Once the virus's genome is inside the host cell, the viral genome takes command of the cell's production machinery and causes the host cell to generate copies of the virus. As the viral copies exit the host cell, these virions set off in search of other host cells to infect.

Naturally occurring viruses exist in a number of differing shapes. The shape of a virus may be rod or filament like, icosahedral, or complex structures combining filament and polygonal shapes. Viruses generally have their outer wall comprised of a protein coat or an envelope comprised of lipids.

An outer envelope comprised of lipids may be in the form of one or two phospholipid layers. When the outer envelope is comprised of two phospholipid layers this is termed a lipid bilayer. For purposes of this text the term ‘lipid’ includes ‘phospholipid’ molecules. A phospholipid is a composite molecule comprised of a polar or hydrophilic region on one end and a nonpolar or hydrophobic region on the opposite end. A lipid bilayer covering a virus, like the membrane of a cell, is constructed with the hydrophilic region of one of the phospholipid layers pointed toward the exterior of the virion and the hydrophilic region of the second phospholipid layer pointed inward toward the center of the virus virion; with the hydrophobic regions of each of the two lipid layers pointed toward each other. The outer envelope of some forms of virus may be comprised of an outer lipid layer or lipid bilayer affixed to a protein matrix for support, the protein matrix being located closer to the center of the virus virion than the lipid layer or lipid bilayer.

Spherical viruses are generally spherical in shape and may be comprised of an outer envelope and one inner shell or alternatively an outer envelope and multiple inner shells. Inner shells are approximately spherical in shape; this is because the proteins comprising the protein matrix shell have an irregular shape to their structure, but when constructed together form a shape that resembles a sphere. In the case of a spherical virus with an outer envelope and one inner shell, the inner shell is often referred to as a nucleocapsid shell comprised of numerous capsid proteins attached to each other. In the case of a spherical virus being comprised of an outer envelope and multiple inner shells, the outermost inner viral shells may be referred to as comprised of a quantity of matrix proteins, where the innermost shell is referred to as a nucleocapsid and is comprised of a quantity of capsid proteins. The inner protein shells are nested inside each other. The cavity created by the innermost shell or nucleocapsid is referred to as the ‘core’ or ‘center of the virus’. Any payload carried by the virus virion is generally carried in the core or center of the virion.

Viruses carry genetic material in the form of deoxyribonucleic acid (DNA) or ribonucleic acid (RNA) as their payload. DNA or RNA genome payloads are carried in the cavity of the nucleocapsid referred to as the core. A virus is therefore generally considered to be a DNA virus if its genome is comprised of DNA or the virus is considered a RNA virus if its genome is comprised of RNA that acts as genetic instructions to generate copies of the virus. Viruses may also carry enzymes as part of their payload. An enzyme such as ‘reverse transcriptase’ transforms a RNA viral genome into DNA. Protease enzymes modify the viral genome once it has entered a host cell. An integrase enzyme assists a DNA viral genome with insertion into the host cell's nuclear DNA. The entire genetic payload is carried inside cavity created by the virus's nucleocapsid shell.

The probes attached to the exterior of a virus are constructed to engage specific cell-surface receptors on a specific type of cell in the body. Only a cell that expresses cell-surface receptors that are capable of being engaged by the probes of a specific virus can act as a host for the virus. Viruses generally use two probes to access a host cell. The first probe makes an initial attachment to the host cell, while the action of the virus's second probe often in conjunction with the action of the first probe cause an access portal to be created in the host cell's exterior plasma membrane. Once an access portal is formed, the virus inserts the contents of its payload into the host cell utilizing the open access portal. Certain types of virus may be engulfed whole by a target cell. Once the virus's genome is inside the cytoplasm of the host cell, any enzymes that accompanied the viral genome into the cell, may begin to modify or assist the virus's genome with infecting and taking control of the host cell's biologic functions.

Probes are attached to the exterior envelope of a virus virion. Probes may be in the form of a protein structure or may be in the form of a glycoprotein molecule. For viruses constructed with a protein matrix as its outer envelope, the probes tend to be protein structures. A portion of the protein structure probe is fixed or anchored in the protein matrix, while a portion of the protein structure probe extends out and away from the protein matrix. The portion of the protein structure probe extending out away from the virus virion is referred to as the ‘exterior domain’, the portion anchored in the protein matrix is the ‘transcending domain’. Some protein probes have a third segment that extends through the envelope and exists inside the virus virion, which is referred to as the ‘interior domain’. The exterior domain of a protein structure probe is intended to engage a specific cell-surface receptor on a biologically active cell the virus is targeting as its host cell.

Viruses that utilize a lipid layer as the outer envelope, are constructed with probes that tend to be glycoproteins. A glycoprotein is comprised of a protein segment and a carbohydrate segment. The carbohydrate segment of the glycoprotein molecule is fixed or anchored in the lipid layer of the outer envelope, while the protein segment extends outward and away from the outer envelope. The protein portion of a glycoprotein probe that extends outward and away from the outer envelope of a virus virion is intended to engage a cell-surface receptor on a biologically active cell the virus is targeting as its host cell.

Some forms of viruses that utilize a lipid layer as its envelope use protein structure probes. In this case, the portion of the protein structure probe that extends outward and away from the outer envelope is the ‘exterior domain’, the portion that is anchored in the lipid layer is the ‘transcending domain’ and again some protein structure probes have an ‘interior domain’ that exist inside the virion, which may also help anchor the protein structure probe to the virion. The exterior domain of a protein structure probe that extends outward and away from the outer envelope of a virus virion is intended to engage a cell-surface receptor on a biologically active cell the virus is targeting as its host cell.

When a virus carries a DNA payload and the viral DNA is inserted into the host cell, the virus's DNA travels to the host cell's nucleus and is known to become inserted into the host cell's own native DNA. In the case where a virus is carrying its genetic payload as RNA, the virus inserts the RNA payload into the host cell and may also insert one or more enzymes to facilitate the RNA being utilized properly to replicate copies of the virus. Once inside the host cell, some species of virus facilitate use of the viral RNA by having the RNA converted to DNA. Once the viral RNA has been converted to DNA, the virus's DNA travels to the host cell's nucleus and is known to become inserted into the host cell's native DNA. Once a virus's genetic material has been inserted into the host cell's native DNA, the virus's genetic material takes command of certain cell functions and redirects the resources of the host cell to generate copies of the virus. Other forms of RNA viruses bypass the need to use the nuclear DNA and simply utilize portions of the viral genome to act as messenger RNA. RNA viruses that bypass the host cell's DNA, cause the cell in general to generate copies of the necessary parts of the virus directly from the virus's RNA genome.

The human immunodeficiency virus (HIV) is a RNA virus and has an outer envelope comprised of a lipid bilayer. The lipid bilayer covers a protein matrix consisting of p17^{gag} proteins. Inside the p17^{gag} protein is nested a nucleocapsid comprised of p24^{gag} proteins. Inside the nucleocapsid HIV carries its payload. HIV's genetic payload consists of two single strands of RNA and several enzymes. The enzymes that accompany HIV's genome include ‘reverse transcriptase’, ‘Integrase’ and ‘protease’ molecules.

The T-Helper cell acts as HIV's host cell. The HIV virion utilizes two types of glycoprotein probes affixed to its exterior envelope to locate and engage a T-Helper cell. HIV utilizes a glycoprotein probe 120 to locate a CD4 cell-surface receptor on a T-Helper cell. Once an HIV glycoprotein 120 probe has successfully engaged a CD4 cell surface-receptor on a T-Helper cell a conformational change occurs in the glycoprotein 120 probe and a glycoprotein 41 probe is exposed. The glycoprotein 41 probe's intent is to engage a CXCR4 or CCR5 cell-surface receptor on the same T-Helper cell. Once a glycoprotein 41 probe on the HIV virion successfully engages a CXCR4 or CCR5 cell-surface receptor, the HIV virion opens an access portal through the T-Helper cell's outer membrane.

Once the HIV virion has opened an access portal through the T-Helper cell's outer plasma membrane, the HIV virion inserts two positive strand RNA molecules and the associated enzymes it carries into the T-Helper cell. Each RNA strand is approximately 9500 nucleotides in length. Inserted along with the RNA strands are the enzymes reverse transcriptase, protease and integrase. Once the virus's genome gains access to the interior of the T-Helper cell, in the cytoplasm the pair of RNA molecules are transformed to deoxyribonucleic acid by the reverse transcriptase enzyme. Following modification of the virus's genome to DNA, the virus's genetic information migrates to the host cell's nucleus. In the nucleus, with the assistance of the integrase protein, the HIV's DNA becomes inserted into the T-Helper cell's native nuclear DNA. When the timing is appropriate, the now integrated viral DNA is decoded by the host cell's polymerase molecules and the virus's genetic information commands certain cell functions to carry out the replication process to construct copies of the human immunodeficiency virus.

The outer layer of the HIV virion is comprised of a portion of the T-Helper cell's outer cell membrane. In the final stage of the replication process, as a copy of the HIV virion, carrying the HIV genome, buds through the host cell's cell membrane the outer protein shell acquires as its exterior envelope, a wrapping of lipid bilayer from the host cell's cell membrane. In the case of HIV, since the surface of the pathogen is covered by an envelope comprised of lipid bilayer taken from the host T-Helper cells, this feature allows the HIV virion the capacity to elude the two immune systems, since the detectors comprising the innate immune system and the adaptable immune system may find it difficult to distinguish between the surface of an infectious HIV virion and the surface characteristics of a noninfected T-Helper cell.

The Hepatitis C virus (HCV) is a positive sense RNA virus, meaning a type of RNA that is capable of bypassing the need for involving the host cell's nucleus by having its RNA genome function as messenger RNA. Hepatitis C infects liver cells. The Hepatitis C viral genome becomes divided once it gains access to the interior of a liver host cell. Portions of the subdivisions of the Hepatitis C genome directly interact with ribosomes to produce proteins necessary to construct copies of the virus.

HCV belongs to the Flaviviridae family and is the only member of the Hepacivirus genus. There are considered to be at least 100 different strains of Hepatitis C virus based on genome sequencing variability.

HCV is comprised of an outer lipoprotein envelope and an internal nucleocapsid. The genetic payload is carried within the nucleocapsid. In its natural state, present on the surface of the outer envelope of the Hepatitis C virus are probes that detect receptors present on the surface of liver cells. The glycoprotein El probe and the glycoprotein E2 probe have been identified to be affixed to the surface of HCV. The E2 probe binds with high affinity to the large external loop of a CD81 cell-surface receptor. CD81 is found on the surface of many cell types including liver cells. Once the E2 probe has engaged the CD81 cell-surface receptor, cofactors on the surface of HCV's exterior envelope engage either or both the low density lipoprotein receptor (LDLR) or the scavenger receptor class B type I (SR-BI) present on the liver cell in order to effect the mechanism to facilitate HCV breaching the cell membrane and inserting its RNA genome payload through the plasma cell membrane of the liver cell into the liver cell. Upon successful engagement of the HCV surface probes with a liver cell's cell-surface receptors, HCV inserts the single strand of RNA and other payload elements it carries into the liver cell targeted to be a host cell. The HCV RNA genome then interacts with enzymes and ribosomes inside the liver cell in a translational process to produce the proteins required to construct copies of the protein components of HCV. The HCV genome undergoes a method of transcription to replicate copies of the virus's RNA genome. Inside the host, pieces of the HCV virus are assembled together and ultimately loaded with a copy of the HCV genome. Replicas of the original HCV then escape the host cell and migrate the environment in search of additional host liver cells to infect and continue the replication process.

The HCV's naturally occurring genetic payload consists of a single molecule of linear positive sense, single stranded RNA approximately 9600 nucleotides in length. By means of a translational process a polyprotein of approximately 3000 amino acids is generated. This polyprotein is cleaved post translation by host and viral proteases into individual viral proteins which include: the structural proteins of C, E1, E2, the nonstructural proteins NS1, NS2, NS3, NS4A, NS4B, NS5A, NS5B, p7 and ARFP/F protein. Hepatitis C virus's proteins direct the host liver cell to construction copies of the Hepatitis C virus. A membrane associated replicase complex consisting of the virus's nonstructural proteins NS3 and NS5B facilitate the replication of the viral genome. The membrane of the endoplasmic reticulum appears to be the site of protein maturation and viral assembly. Once copies of the Hepatitis C Virus are generated, they exit the host cell and each copy of HCV migrates in search of another appropriate liver cell that will act as a host to continue the replication process.

Hepatitis C virus life-cycle demonstrates that copies of a virus virion can be generated by inserting RNA into a host cell that functions as messenger RNA in the host cell. The Hepatitis C viral RNA genome functions as messenger RNA, acting as the template in conjunction with the biologic machinery of a host cell to produce the components that comprise copies of the Hepatitis C virion and the Hepatitis C viral RNA provides the biologic instructions to assemble the components into complete copies of the Hepatitis C virions. The Hepatitis C virus life-cycle clearly demonstrates that viral virions can be manufactured by a host cell without involving the nucleus of the cell.

Deciphering the existence, replication and behavior of viruses provides clear examples of several fundamental concepts, which include: (1) Viruses target specific cells in the body by means of identifying and engaging such target cells utilizing the probes projecting outward from the virus's exterior shell to make contact with cell-surface receptors located on the surface of their host cells, and (2) Viruses are capable of carrying a variety of different types of payloads including DNA, RNA and a variety of proteins.

Current gene therapy approach to attempting to deliver a payload to cells in the body use modified forms of existing viruses to act as transport devices to deliver genetic information. This approach is severely limited by restricting the virus virion to the target only cells the viral vector naturally seeks out and infects. Current gene therapy approach is further limited by using the pre-existing size of naturally occurring viruses, rather than being able to modify the size of the structure to be able to tailor the volumetric carrying capacity of the payload portion of the modified virus. Further, gene therapy is restricted to utilizing naturally occurring viruses to deliver only genetic information; it has not previously been appreciated by those skilled in the art that virus-like transport devices might deliver to a variety of specific cell types a wide variety of differing payloads.

Ribonucleic acids are inherent to a cell and RNAs are used by some viruses to act as the virus's genome to generate copies of the virus. RNAs inherent to a cell are referred to as ‘cellular RNAs’ and include protein coding RNAs and non-coding RNAs (ncRNA). Protein coding RNAs, generally referred to as messenger RNAs, code for proteins and undergo translation to produce protein molecules. Non-coding RNA represent a variety of functional RNA molecules that do not undergo translation to produce a protein. Non-coding RNAs are highly abundant and functionally important for the cell. Non-coding RNAs have also referred by such terms as non-protein-coding RNAs (npcRNA) or non-messenger RNA (nmRNA) or small non-messenger RNA (snmRNA) or functional RNAs (fRNA). The non-coding RNAs include: transfer RNAs (tRNA), ribosomal RNAs (rRNA), small nuclear RNAs (snRNA), small nucleolar RNAs (snoRNA), signal recognition particle RNA (SRP RNA), antisense RNA (aRNA), micro RNA (miRNA), small interfering RNA (siRNA), Y RNA, telomerase RNA. RNA found in naturally occurring viruses are referred to as ‘viral RNA’.

Transfer RNAs (tRNA), are RNAs that carries amino acids and deliver them to a ribosome. Ribosomal RNAs (rRNA), are RNAs that couple with ribosomal proteins and participate in translation of mRNA to produce protein molecules. Small nuclear RNAs (snRNA) are RNAs involved in splicing and other nuclear functions. Small nucleolar RNAs (snoRNA) are RNAs involved in nucleotide modification. Signal recognition particle RNA (SRP RNA) are RNAs are involved in membrane integration. Antisense RNA (aRNA) are RNAs involved in transcription attenuation, mRNA degradation, mRNA stabilization, and translation blockage. Micro RNA (miRNA) are RNAs involved in gene regulation and have been implicated in a wide range of cell functions including cell growth, apoptosis, neuronal plasticity, insulin secretion. Small interfering RNA (siRNA) are RNAs involved in gene regulation, often interfering with the expression of a single gene. Y RNA are RNAs involved in RNA processing and DNA replication. Telomerase RNA are RNAs involved in telomere synthesis. The primary purpose of ‘viral RNA’ is to make copies of the virus that carries of a genome RNA.

Messenger RNA molecules are comprised of three regions (or segments). These three regions include: (1) a 5′ untranslatable region, (2) a coding region and (3) a 3′ untranslatable region. The 5′ untranslatable region acts as the initiation point for a ribosome to attach to the mRNA. The ‘coding region’ acts as the template from which a protein is constructed. An ‘untranslatable region’ represents a segment of a messenger RNA molecule that does not code for a protein and is not used to yield a protein and therefore ‘translation’ does not occur in such a region. The 3′ untranslatable region is associated with the degradation of the usefulness of the mRNA. Different mRNAs have different service life expectancies. The half-life of the naturally occurring mRNA that acts as the template responsible for the production of the protein ‘glucokinase’ is two hours. The half-life of the naturally occurring mRNA that acts as the template to produce the protein ‘alcohol dehydrogenase’ is ten hours. The half-life of the naturally occurring mRNA that acts as the template to produce the protein ‘glucuronidase’ is thirty hours. By modifying the nucleotides that comprise the 3′ untranslatable unit of an mRNA, the service half-life of the mRNA may be altered to be lengthened or shortened depending upon the need for the quantity of protein and timeframe over which the mRNA is required to produce the protein coded in the protein template of the mRNA's coding region.

RNA found in naturally occurring viruses are referred to as ‘viral RNA’. The primary function of ‘viral RNA’ is dedicated to making copies of the virus that carries the RNA genome. Cellular RNAs are inherent to a cell and do not include ‘viral RNAs’. Modifications to messenger RNA molecules occur naturally due to errors that occur in the DNA and errors that occurring during the transcription phase and maturation phase of generating messenger RNA. Modifications to ribonucleic acid molecules may occur purposely to produce a medical therapeutic response.

Modified ribonucleic acid molecules are naturally occurring cellular ribonucleic acid molecules that have purposely undergone modification to the nucleotide sequence to enhance the performance of the naturally occurring cellular ribonucleic acid molecule. Naturally occurring cellular ribonucleic acid molecules can purposely have their nucleotide sequence modified in the 3′untranslatable region, the coding region or the 5′ untranslatable region or modification may be made in any combination of the three regions to enhance the performance of the modified ribonucleic acid molecule above and beyond that of the naturally occurring ribonucleic acid molecule. Naturally occurring cellular ribonucleic acid molecules that purposely have a quantity of their nucleotides altered to produce a medical benefit are termed ‘modified ribonucleic acids’, versus naturally occurring cellular ribonucleic acid molecules that undergo mutation due to an error that occurs in production of the molecule would be termed ‘mutant ribonucleic acids’.

Research has demonstrated that natural proteins can be altered to produce medically beneficial effects. The parathyroid hormone (PTH) is one example. Intact PTH is produced by cells in the parathyroid glands. There are four parathyroid glands present in the neck, generally in the vicinity of the thyroid gland. The term ‘para-’ means ‘next to’, so early anatomists identified the four glands ‘parathyroid glands’ because they were generally found ‘next to’ the thyroid gland in the neck. Parathyroid hormone is released in response to the cells of the parathyroid gland sensing a decline in the level of serum calcium. Parathyroid hormone, in its natural state, acts to stimulate osteoclast cells present in bone to release calcium from bone, thereby acting as a mechanism to return the serum calcium level to the normal range whenever the serum calcium drops below the normal range. On the other hand, it has been quite well demonstrated that if (1) the amino acid chain of the parathyroid hormone is shortened and (2) the shorter parathyroid hormone molecule is pulsed, by injecting it into the body once a day, the action of this modified parathyroid hormone molecule is opposite of the intact parathyroid hormone. One such form of a shorter length parathyroid hormone molecule is termed ‘teriparatide’. Teriparatide (1-34) has the identical sequence from 1 to the 34^{th} N-terminal amino acid of the 84-amino acid endogenous human parathyroid hormone. The skeletal effects of the modified protein molecule act on bone cells to preferentially cause osteoblastic activity over osteoclastic activity, which results in storage of calcium into bone, rather than a release of calcium from bone if the teriparatide is administered once a day. Teriparatide has been a recognized and widely used treatment of osteoporosis since at least as far back as the year 2000.

Purposely modifying the ‘coding region’ of a messenger RNA modifies the protein the messenger RNA produces when ribosomes attach to and translate such a modified messenger RNA. As demonstrated by the case of modifying the naturally occurring parathyroid hormone by administering a molecule that is comprised of fewer amino acids than the original PTH molecule, modifying proteins the messenger RNAs produce may provide health care providers with an entirely new and widely spanning armamentarium of medically beneficial therapies.

The 5′ untranslatable region of a messenger RNA molecule is used to identify the messenger RNA and utilized as a point of attachment by ribosomes to the messenger RNA molecule. Modifying the 5′ untranslatable region of a messenger RNA by altering the nucleotide sequence in the 5′ untranslatable region makes it easier to identify a modified messenger ribonucleic acid molecules in a fashion that the modified messenger ribonucleic acid molecules can be more easily or readily engaged by ribosomes. Altering the nucleotide sequence of the 5′ untranslatable region of a modified messenger ribonucleic acid molecule to create a unique identifier, facilitates ribosomes to preferentially engage the modified messenger ribonucleic acid molecule to preferentially produce the protein for which the modified messenger ribonucleic acid molecule is acting as a template.

Modifying the nucleotide sequence of messenger ribonucleic acid molecules in the 3′ untranslatable region extends or shortens the service life of said modified messenger ribonucleic acid molecules, compared to naturally occurring messenger ribonucleic acid molecules. Service life refers to the quantity of time a messenger RNA molecule, present in the cytoplasm, will be able to undergo translation before it is degraded by cellular enzymes. By modifying the nucleotide sequence of a messenger RNA molecule to extend its service life, this allows additional time for ribosomes to decode the information present on the modified messenger ribonucleic acid molecules. Modifying the naturally occurring messenger ribonucleic acid molecule in the 3′ untranslatable region in a fashion to cause the molecule to resist degradation by cellular enzymes without compromising the functionality of the modified messenger ribonucleic acid molecules increases production of proteins by ribosomes which results in an enhanced medically therapeutic treatment.

A dramatic, not previously recognized by those expert in the art is the need to develop a transport vehicle that can be fashioned to seek out specific types of cells and deliver to these cells cellular ribonucleic acid molecules to treat medical conditions related to a protein deficiency. By providing cellular RNAs to these specific target cells a wide variety of cellular functions can be enhanced including gene expression, protein production, and telomere synthesis. The exterior envelope of a transport should be constructed so as not to alert the immune system to its presence to prevent rejection of these vehicles. Transport vehicles should be capable of being configured to target any specific type of cell and engage and deliver their payload only to that specific type of cell. To this point, no such device or process has been conceived.

### BRIEF SUMMARY OF THE INVENTION

Utilization of configurable microscopic medical payload delivery devices to deliver ribonucleic acid molecules to specific types of cells facilitates a dramatic new approach to managing a wide variety of medical conditions. By selecting the type of probes that will effectively engage cell-surface receptors on target cells and fixing these probes on the surface of the configurable microscopic medical payload delivery devices, specific types of cells can be targeted. By utilizing configurable microscopic medical payload delivery devices to deliver cellular ribonucleic acid molecules to specific cell types, medical conditions including protein deficient states, genetic deficiencies, conditions related to over expression of genes previously incapable of being treated, are now treatable utilizing this new and unique approach.

### DETAILED DESCRIPTION

The future of medical treatment includes the aggressive, widespread utilization of configurable microscopic medical payload delivery devices (CMMPDD) to deliver a wide variety of ribonucleic acid molecules directly to targeted cell types in the body.

This patent introduces the concepts: (1) configurable microscopic medical payload delivery devices can carry ribonucleic acid molecules inherent to the cell as the payload, and (2) glycoprotein probes present on the exterior of the configurable microscopic medical payload delivery devices include specific glycoprotein probes or protein structure probes affixed to the exterior, these glycoprotein probes or protein structure probes intended to seek out and engage cell-surface receptors attached to the exterior of whichever cell the configurable microscopic medical payload delivery devices is intended to deliver its payload of cellular RNAs to in order to produce a predetermined medically beneficial effect.

For purposes of this text, the use of the terms ‘specific target cell type’, ‘target cell’, ‘specific cell type’, ‘specific cell’, ‘specific type of cell’ are equivalent and interchangeable; the configuration of cell-surface receptors that a specific cell type has located on and protruding from its outer cell membrane determines the cell type.

For purposes of this text an ‘external envelope’ refers to the outermost covering of a virus or a virus-like transport device or a configurable microscopic medical payload delivery device. The external envelope may be comprised of a lipid layer, a lipid bilayer, the combination of a lipid layer affixed to a protein matrix or the combination of a lipid bilayer affixed to a protein matrix. A protein matrix is equivalent to a protein shell and may be referred to as a protein matrix shell. The terms protein matrix, protein shell, protein matrix shell are equivalent to the term capsid, where the term capsid is meant to represent ‘a protein coat or shell of a virus particle, surrounding the nucleic acid or nucleoprotein core’. For purposes of this text, the term ‘particle’ is equivalent to the term ‘virion’; further the term ‘virus particle’ is equivalent to ‘viral virion’.

For purposes of this text an ‘internal shell’ refers to a protein matrix shell nested inside the external envelope. Multiple inner shells may exist, with those of smaller diameter concentrically nested inside those of a larger diameter. The innermost protein matrix shell is termed the nucleocapsid. The proteins that comprise the nucleocapsid are termed capsid proteins. In the cavity created by the nucleocapsid, referred to as the center or core of the nucleocapsid, is where the payload of ribonucleic acid molecules is carried.

For purposes of this text ‘external probes’ are molecular structures that are utilized to locate and engage cell-surface receptors on biologically active cells. External probes are generally comprised of a portion which is anchored or fixed in the external envelope and a second portion that extends out and away from the external envelope. The portion of the external probe that extends out and away from the external envelope is intended to make contact and engage a specific cell-surface receptor located on a biologically active cell. External probes may be comprised solely of a protein structure or an external probe may be a glycoprotein molecule.

For purposes of this text ‘glycoprotein molecule’ refers to a molecule comprised of a carbohydrate region and a protein region. Glycoprotein molecules that act as probes are generally anchored or fixed to a lipid layer utilizing the carbohydrate portion of the molecule as an anchor. The protein portion of the glycoprotein molecule which extends outward and away from the exterior envelope the glycoprotein has been affixed such that the protein region may function as a probe to locate and attach to the cell-surface receptor it was created to engage.

The concept of configurable microscopic medical payload delivery devices is modeled after naturally existing viruses. Configurable microscopic medical payload delivery devices in general are spherical in shape; though other shapes may be used as function might warrant the use of a particular shape. The spherical configurable microscopic medical payload delivery devices are comprised of an exterior envelope and one or more nested inner protein shells. A quantity of exterior protein structure probes and/or glycoprotein probes are anchored in the exterior envelope and a portion extends out and away from the exterior envelope. Nesting of protein shells refers to progressively smaller diameter shells fitting snugly inside protein shells of a larger diameter. Inside the innermost protein shell, referred to as the nucleocapsid, is a cavity referred to as the core of the device. The core of the device is the space where the medically therapeutic payload the device carries is located. The payload of the device is comprised of ribonucleic acid molecules.

Configurable microscopic medical payload delivery devices (CMMPDD) target specific types of cells in the body. Configurable microscopic medical payload delivery devices engage specific types of cells by the configuration of probes affixed to the exterior envelope of the CMMPDD. By fixing specific probes to the exterior envelope of the CMMPDD, these probes intended to engage and attach only to specific cell-surface receptors located on certain cell types in the body, the CMMPDD will deliver its payload to only those cell types that express compatible and engagable specific cell-surface receptors. In a similar fashion where the exterior probes of a naturally occurring virus engage specific cell-surface receptors present on the surface of the virus's host cell and only the designated host cell, the CMMPDD's exterior probes are configured to engage cell-surface receptors on a specific type of target cell and only those cells. In this manner, the payload of cellular RNAs carried by CMMPDD will be delivered only to specific types of cells in the body. The configuration of the exterior probes on the surface of a CMMPDD varies as needed so as to effect the CMMPDD delivery of specific cellular RNA payloads to specific types of cells as needed to effect a particular predetermined medical treatment.

The size of the configurable microscopic medical payload delivery devices is dependent upon the diameter of the inner protein matrix shells and this is dictated by the volume size of the payload the CMMPDD is required to carry and deliver to a target cell. The diameter of each inner protein matrix shell is governed by the number of protein molecules utilized to construct the protein matrix shell at the time the protein matrix shell is generated. Increasing the number of proteins that comprise a protein matrix shell increases the diameter of the protein matrix shell. When applicable, as dictated by the capacity the CMMPDD is to be utilized to function as, an external lipid envelope wraps around and covers the outermost protein matrix shell. The larger the volume of the core of the CMMPDD, the greater the physical size of the payload the CMMPDD is able to carry. The size of the configurable microscopic medical payload delivery device is to be generally the size of cell (approximately 10⁻⁴ m in diameter) or less, generally detectable by a light microscope or, as needed, an electron microscope. The size of the CMMPDD is not to be too large such that it would generate a burden to the body by damaging organ tissues through clogging blood vessels or the glomeruli in the kidneys. The dimensions of each type of CMMPDD are to be tailored to the mission of the CMMPDD, which takes into account factors such as the type of target cell, the size of the payload that is to be delivered to the target cells and the length of time the CMMPDD may have to engage the target cell.

The payload of the configurable microscopic medical payload delivery devices include cellular RNAs, which include protein coding RNAs and non-coding RNAs. Protein coding RNAs include messenger RNAs. The non-coding RNAs include: transfer RNAs (tRNA), ribosomal RNAs (rRNA), small nuclear RNAs (snRNA), small nucleolar RNAs (snoRNA), signal recognition particle RNA (SRP RNA), antisense RNA (aRNA), micro RNA (miRNA), small interfering RNA (sRNA), Y RNA, telomerase RNA.

Being enveloped in an external lipid layer, configurable microscopic medical payload delivery devices possess the advantage of having their exterior appear similar to the plasma membrane that acts as an outside covering for the cells that comprise the body. By appearing similar to existing plasma membranes, the CMMPDDs appear similar to naturally occurring structures found in the body. CMMPDD are afforded the capability to avoid detection by a body's immune system because the exterior of the CMMPDD mimics the cells comprising the body and the surveillance elements of the immune system find it difficult to discern between the CMMPDD and naturally occurring cells comprising the body.

To carry out the process of manufacturing a configurable microscopic medical payload delivery device, a primitive cell such as a stem cell is selected. The reason for utilizing primitive cells such as stems cells as the host cell, is that the CMMPDD acquires its outer envelope from the host cell and the more primitive the host cell, the fewer in number the identifying protein markers are present on the surface of the CMMPDD. The fewer the identifying surface proteins present on the outer envelope of the CMMPDD, the less likely a body's immune system will identify the CMMPDD as an intruder and therefore less likely the body's immune system will react to the presence of the CMMPDD and reject the CMMPDD by attacking and neutralizing the CMMPDD.

Stem cells used as host cells to manufacture quantities of CMMPDD product are selected per histocompatibility markers present on their surface. Certain histocompatibility markers present on the surface of the final CMMPDD product will be less likely to cause a reaction in a specific patient based on the genetic profile of the patient's histocompatibility markers. A similar histocompatibility match is done when donor organs are selected to be given to recipients to avoid rejection of the donor organ by the recipient's immune system.

The selected stem cells used to manufacture configurable microscopic medical payload delivery devices goes through several steps of maturation before it is capable of generating therapeutic CMMPDD product. RNA inserted into the host stem cell code for the general physical outer structures of the CMMPDD. RNA inserted into the host generate surface probes that target the cell-surface receptors on a specific target cell type. RNA is inserted into the host that is used to generate the payload of ribonucleic acid molecules. Similar to how copies of a naturally occurring virus, such as the Hepatitis C virus or HIV, are produced, assembled and released from a host cell, copies of the CMMPDD are produced, assembled and released from a stem cell functioning as a de facto host cell. Once released from the host cell, the copies of the CMMPDD are collected, then pooled together to produce a therapeutic dose that results in a medically beneficial effect.

The stem cells used as host cells are suspended in a broth of nutrients and are kept at an optimum temperature to govern the rate of production of the CMMPDD product. Similar to the natural production of the Hepatitis C virus, the configurable microscopic medical payload delivery devices ‘production genome’ is introduced into the host stem cells. The configurable microscopic medical payload delivery devices production genome carries genetic instructions to cause the host cells to manufacture the configurable microscopic medical payload delivery devices' outer protein wall, the inner protein matrixes, the surface probes the configurable microscopic medical payload delivery device is to have affixed to its outer envelope, the ribonucleic acid molecules the configurable microscopic medical payload delivery devices are to carry, and the instructions to assemble the various pieces into the final form of the configurable microscopic medical payload delivery devices along with the instructions to activate the budding process. The resultant configurable microscopic medical payload delivery devices are collected from the nutrient broth surrounding the host cells and placed together into doses to be used as a treatment for a protein deficiency state.

The ‘production genome’ are an array of RNAs, which include messenger RNAs that are directly translated by the host cell's ribosomes. The production genome dictates the characteristics of the final version of the CMMPDD that buds from the host stem cell and is released and is to be utilized as a medical treatment. The production genome is specifically tailored to code for the surface probes that will seek and engage a specific type of target cell. The production genome also carries the instructions to code for the production of the type of ribonucleic acid molecules to be delivered to the specific type of target cell. The ‘production genome’ varies depending upon the configuration of the CMMPDD and the specific type of ribonucleic acid molecules the CMMPDD will transport to effect a specific predetermined medical treatment in a specific type of cell.

The configurable microscopic medical payload delivery device transporting ribonucleic acid molecules represents a very versatile medical treatment delivery device. CMMPDD is used to deliver a number of different ribonucleic acid molecules to a wide variety of cells in the body.

The construction of a naturally occurring virus can be likened to the act of following a programmed script to produce a specific result. It is known that the genetic code that a virus carries dictates the production of copies of the virus. It is known that specific segments of the viral genetic code represent instructions that dictate the construction of different parts of the virus so that copies of the virus can be made inside the host cell. It is well documented that there exist different subtypes of most viruses, based off of mutations that have occurred to the viral genome over time; these mutations to the viral genome producing variants in the construction of the virus. Configurable microscopic medical payload delivery devices which carry RNA are constructed much like a naturally occurring virus virion would be constructed in a host cell. Altering the production RNA alters the configuration of the external probes or alters the configuration of the size of the inner shells or alters the type of RNA the CMMPDD will carry or alters any combination of the three.

As an example of this method, to treat diabetes mellitus the following production process is followed in the lab: (1) Human stem cells are selected. (2) Into the selected stem cells is placed the RNA production genome constructed, in this case, specifically as a means to treat diabetes mellitus. The RNA production genome contains genetic instructions to cause the host stem cells to manufacture the CMMPDDs' outer protein wall, the inner protein matrix, surface probes to include a quantity of glycoprotein probes that engage the GPR40 cell-surface receptor present on the surface of Beta cells located in the Islets of Langerhans in the pancreas, and the messenger RNA payload to facilitate the production of the insulin molecule; and the biologic instructions to assemble the components into the final form of the CMMPDD and the biologic instructions to activate the budding process. (3) Upon insertion of the RNA production genome into the host stem cells, host stem cells' protein production cellular machinery responds by simultaneously translating the different segments of the RNA production genome to produce the proteins that comprise the exterior protein wall, the inner protein matrix molecules, the surface probes, the mRNA payload to produce insulin and decode the instructions to assemble the components into the CMMPDDs. (4) Upon assembly, the CMMPDDs bud through the cell membrane of the host stem cell. (5) At the time of the budding process, the CMMPDDs acquire an outside envelope over the outer protein shell, this outer envelope comprised of a portion of the plasma membrane from the host stem cell the CMMPDD exits. (6) The resultant CMMPDDs are collected from the nutrient broth surrounding the host stem cells. (7) The CMMPDD product is washed in a sterile solution to remove unwanted elements of the nutrient broth. (8) The configurable microscopic medical payload delivery devices are removed from the sterile solution wash and suspended in a sterile hypoallergenic liquid medium. (9) The CMMPDD are separated into individual quantities to facilitate storage and delivery to physicians and patients. (10) The CMMPDD product carried in the sterile hypoallergenic liquid medium is administered to a diabetic patient per injection in a dose that is tailored to receiving patient's requirement to produce sufficient amount of insulin to control the blood sugar. (11) Upon being injected into the body, the CMMPDD product migrates to the Beta cells located in the Islets of Langerhans by means of the blood stream. (12) Upon the CMMPDD product reaching the Beta cells, the probes on the surface of the CMMPDDs engage the cell-surface receptors located on the Beta cells and inserts the RNA payload, including mRNA, into the Beta cells. The mRNA payload is translated by the cell's ribosomes to produce insulin molecules. The increase in insulin production by Beta cells successfully treats diabetes mellitus.

In a similar fashion, configurable microscopic medical payload delivery devices can be fashioned to deliver a payload of a specific type of ribonucleic acid molecule to any type of cell in the body. Different cell types express different cell-surface markers on the exterior of their plasma membrane. The differing configurations of cell-surface markers on differing types of cells distinguish one cell type from another cell type. By configuring the exterior probes that extend from the surface of the configurable microscopic medical payload delivery device to seek out and engage specific cell-surface receptors present on a specific cell type, payloads of any messenger ribonucleic acid molecule or any non-coding ribonucleic acid molecule can be delivered to specific cells in the body.

### Conclusions, Ramification, and Scope

Accordingly, the reader will see that the configurable microscopic medical payload delivery device to deliver cellular ribonucleic acid molecules to specific targeted cell types provides advantages over existing art by (1) being a delivery device that seeks out specific types of cells, (2) by being a delivery device that is versatile enough to deliver a variety of cellular ribonucleic acid molecules to accomplish various medical treatments and (3) by being a delivery device constructed with a surface envelope that will avoid detection by the innate immune system and the adaptable immune system so as not to activate the immune system to its presence; for these reasons this represents a new and unique medical delivery device that has never before been recognized nor appreciated by those skilled in the art.

Although the description above contains specificities, these should not be construed as limiting the scope of the invention but as merely providing illustrations of some of the presently preferred embodiments of the invention.

Thus the scope of the invention should be determined by the appended claims and their legal equivalents, rather than by the examples given.

What is claimed:

1. A configurable microscopic medical payload delivery device comprised of:
(a) an exterior envelope,
(b) a quantity of interior shells,
(c) a quantity of configurable exterior probes attached in a manner a segment of said exterior probes is to project out and away from said exterior envelope, while a segment of said exterior probes is embedded in said exterior envelope, and
(d) a medically therapeutic payload comprised of a quantity of cellular ribonucleic acid molecules,
whereby said configurable microscopic medical payload delivery device is intended to deliver said quantity of cellular ribonucleic acid molecules to a specific type of cell in order to produce a medically beneficial effect,
whereby said exterior probes are intended to engage specific cell-surface receptors on said specific type of cell,
whereby said cellular ribonucleic acid molecules are carried as said medically therapeutic payload in the core of said configurable microscopic medical payload delivery device,
whereby said interior shells are versatile enough in their construction to transport within the cavity created by the nucleocapsid a plurality of said cellular ribonucleic acid molecules as said medically therapeutic payload.

2. The configurable microscopic medical payload delivery device in claim 1 wherein said external envelope is selected from the group consisting of a lipid layer, a lipid bilayer, a protein matrix, a lipid layer affixed to a protein matrix, and a lipid bilayer affixed to a protein matrix.

3. The configurable microscopic medical payload delivery device in claim 1 wherein said external envelope is comprised of a quantity of lipid layers and a quantity of protein matrix shells.

4. The quantity of lipid layers in claim 3 wherein said quantity of lipid layers is a quantity of phospholipid layers.

5. The configurable microscopic medical payload delivery device in claim 1 wherein said quantity of internal shells are comprised of a quantity of nested sphere-like concentric protein matrix shells.

6. The configurable microscopic medical payload delivery device in claim 1 wherein said exterior probes are comprised of a quantity of protein structure probes and a quantity of glycoprotein probes.

7. The protein structure probes in claim 6 wherein said protein structure probes are comprised of a segment of said protein structure probes which extends outward and away from said exterior envelope, which is attached to a segment of said protein structure probes which is embedded in said exterior envelope to hold said protein structure probes affixed to said exterior envelope,
whereby said segment of said protein structure probes which extends outward and away from said exterior envelope is intended to engage said specific cell-surface receptors on said specific type of cell.

8. The protein structure probes in claim 6 wherein said protein structure probes are comprised of a plurality of protein structure probes,
whereby, at least two differing configurations of said protein structure probes may be needed to successfully engage said specific type of cell with one type of configuration of said protein structure probe engaging one type of said specific cell-surface receptor, while a differing type of configuration of said protein structure probe is required to engage a differing type of said specific cell-surface receptor in order for said configurable microscopic medical payload delivery device to insert said quantity of cellular ribonucleic acid molecules said configurable microscopic medical payload delivery device carries into intended said specific type of cell.

9. The glycoprotein probes in claim 6 wherein said glycoprotein probes are comprised of a protein segment, which extends outward and away from said exterior envelope, which is attached to a carbohydrate segment, said carbohydrate segment embedded in said exterior envelope to hold said glycoprotein probe affixed to said exterior envelope,
whereby said protein segment which extends outward and away from said exterior envelope is intended to engage said specific cell-surface receptor on said specific type of cell.

10. The glycoprotein probes in claim 6 wherein said glycoprotein probes are comprised of a plurality of glycoprotein probes,
whereby, at least two differing configurations of said glycoprotein probes may be needed to successfully engage said specific type of cell with one type of configuration of said glycoprotein probes engaging one type of said specific cell-surface receptor, while a differing type of configuration of said glycoprotein probes is required to engage a differing type of said specific cell-surface receptor in order for said configurable microscopic medical payload delivery device to insert said quantity of cellular ribonucleic acid molecules into said specific type of cell.

11. The cellular ribonucleic acid molecules in claim 1 wherein said cellular ribonucleic acid molecules comprise a quantity of messenger ribonucleic acid molecules and a quantity of non-coding ribonucleic acid molecules.

12. The non-coding ribonucleic acid molecules in claim 11 wherein said non-coding ribonucleic acid molecules are comprised of a quantity of transport ribonucleic acid molecules, a quantity of ribosomal ribonucleic acid molecules, a quantity of small nuclear ribonucleic acid molecules, a quantity of small nucleolar ribonucleic acid molecules, a quantity of signal recognition particle ribonucleic acid molecules, a quantity of antisense ribonucleic acid molecules, a quantity of micro ribonucleic acid molecules, a quantity of small interfering ribonucleic acid molecules, a quantity of Y ribonucleic acid molecules, and a quantity of telomerase ribonucleic acid molecules.

13. The messenger ribonucleic acid molecules in claim 11 wherein said messenger ribonucleic acid molecules are comprised of a quantity of naturally occurring messenger ribonucleic acid molecules and a quantity of modified messenger ribonucleic acid molecules.

14. The modified messenger ribonucleic acid molecules in claim 13 wherein said modified ribonucleic acid molecules is comprised of a quantity of messenger ribonucleic acid molecules modified in the 3′ untranslatable region, a quantity of messenger ribonucleic acid molecules modified in the coding region, and a quantity of ribonucleic acid molecules modified in the 5′ untranslatable region,
whereby said modification of said messenger ribonucleic acid molecules in said 3′ untranslatable region results in said modified messenger ribonucleic acid molecules resisting degradation by cellular enzymes without compromising the functionality of said modified messenger ribonucleic acid molecules, which results in enhance protein production by said ribosomes to produce a medically therapeutic treatment,
whereby said modification of said modified messenger ribonucleic acid molecules occurring in said coding region results in production of improved proteins,
whereby said modification of said modified messenger ribonucleic acid molecules in said 5′ untranslatable region results in an enhanced recognition of said modified ribonucleic acid molecules by ribosomes and encourages said modified ribonucleic acid molecules to be translated by said ribosomes.

15. The modified messenger ribonucleic acid molecules in claim 13 wherein said modified ribonucleic acid molecules is comprised of a quantity of messenger ribonucleic acid molecules which are comprised of a quantity of nucleotides modified in the 3′ untranslatable region, a quantity of nucleotides modified in the coding region, and a quantity of nucleotides modified in the 5′ untranslatable region,
whereby said modification of said messenger ribonucleic acid molecules in said 3′ untranslatable region results in said modified messenger ribonucleic acid molecules resisting degradation by cellular enzymes without compromising the functionality of said modified messenger ribonucleic acid molecules, which results in enhance protein production by said ribosomes to produce a medically therapeutic treatment,
whereby said modification of said modified messenger ribonucleic acid molecules occurring in said coding region results in production of improved proteins,
whereby said modification of said modified messenger ribonucleic acid molecules in said 5′ untranslatable region results in an enhanced recognition of said modified ribonucleic acid molecules by ribosomes and encourages said modified ribonucleic acid molecules to be translated by said ribosomes.
